(19) United States
(12) Patent Application Publication
(10) Pub. No.: US 2008/0102517 A1
(43) Pub. Date: May 1, 2008

(54) Nanocatalyst-based biosensor

(75) Inventors: Hae Sik Yang, Yeonjae-gu (KR); Jagotamoy Das, Baniyachong (BD)

Correspondence Address: SOFER & HAROUN LLP., 317 MADISON AVENUE, SUITE 910, NEW YORK, NY, 10017, US

(73) Assignee: Pusan National University

(21) Appl. No.: 11/708,390

(22) Filed: Feb. 20, 2007

(30) Foreign Application Priority Data
Oct. 26, 2006 (KR) 10-2006-0104252

(51) Int. Cl. C12M 1/34

(52) U.S. Cl. 435/287.1

(57) ABSTRACT

Disclosed is a biosensor for electrochemically detecting the presence of an analyte and measuring its concentration. In the biosensor, an organic substance as a substrate is reduced to a substance having an amine group by the catalysis of a nanocatalyst label with a plurality of active sites in the presence of a reducing agent, the reduction is continuously carried out to amplify the substance having an amine group, and the amount of the substance having an amine group is measured by taking advantage of the fact that the substance having an amine group exhibits different electrochemical, chromatic or fluorescent properties from those of the substrate, so that the concentration of a biomarker is measured with high sensitivity within a short time. The electrochemical biosensor allows simple measurement of an analyte and has a low detection limit.

### RELATED APPLICATION

This application is related to and claims the benefit of priority from Korean Patent Application No. 10-2006-0.104252, filed on Oct. 26, 2006, the entirety of which is incorporated herein by reference.

### BACKGROUND OF THE INVENTION

1. Field of the Invention

The present invention relates to a biosensor for measuring the concentration of analytes, such as biomolecules, with high sensitivity, and more particularly to a biosensor utilizing the catalysis of nanoparticles.

2. Description of the Related Art

In recent years, there has been a growing need for early diagnosis of diseases to reduce medical expenses and extend human life. In many cases, biomarkers, whose detection indicates a particular disease state, exist at very small concentrations in samples. Under these circumstances, there is a need for a biosensor capable of detecting the presence of biomarkers and measuring their concentrations with high sensitivity. Various methods for increasing the sensitivity of biosensors have been developed to date. Of these, methods for the amplification of signals using enzymes are currently in use.

Enzymes are proteins that selectively catalyze biochemical reactions. Catalysis induced by enzymes is so rapid that it can be utilized to produce signal-generating substances within a short time. When enzymes as labels are attached to biomolecules, which are biospecifically bound to biomarkers, substances formed through the catalysis of the enzymes can be used to identify whether biospecific binding is indeed present or not and to determine the concentration of the biomarkers. A very large number of signal-generating substances per biospecific binding enable large amplification of signals. Enzyme-linked immunosorbent assay is a standard test for the detection and quantification of antigens or antibodies using an enzyme in hospitals. Methods using enzyme labels have drawbacks in that the activity of enzymes used varies with time and the detection limits are as relatively high as 1 pM.

Numerous methods using DNA instead of enzymes have been developed for the amplification of signals. Such signal amplification methods using DNA can be largely classified into the following two groups: i) methods for amplifying DNA as a label after biospecific binding to a biomarker, and ii) methods using previously amplified DNA as a label.

According to the former methods, DNA as a label is amplified by a DNA amplification technique, e.g., polymerase chain reaction (PCR), after biospecific binding to a biomarker (T. Sano, C. Smith, C. Cantor, Science 1992, 258, 120-122). This DNA amplification is performed at a very high rate, which results in much larger amplification than in a method using an enzyme as a label. The amount of the amplified DNA can also be identified in real time by incorporating a fluorescent substance into the amplified DNA to emit fluorescent light. Such a method for amplifying a DNA label is termed ‘immuno-PCR’, by which detection limit as low as 1 fM can be achieved. Since immuno-PCR has the problem that false results may be obtained due to various factors, such as contamination of samples, it has not yet been put to practical use despite low detection limit.

According to the latter methods, when DNA and biomolecules binding to a biomarker are immobilized on magnetic beads as labels, the number of the DNA per biospecific binding is increased by markedly increasing the ratio of the number of the DNA to the number of the biomolecules (U.S. Patent Publication No. 2006/0040286 and J.-M. Nam, C. S. Thaxton, C. A. Mirkin, Science 2003, 301, 1884-1886). An additional amplification of previously amplified DNA by a suitable subsequent amplification technique, such as immuno-PCR, leads to a very low detection limit on the order of aM. However, problems encountered in the methods are that complicated conjugation chemistries of biomolecules and complex measurement procedures are involved.

Further, as taught in J. T. Mason, L. Xu, Z.-M. Sheng, T. J. O'Leary, Nature Biotechnology 2006, 24, 555-557, a very low detection limit can be achieved by encapsulating liposomes as labels with many DNA reporters, rupturing the liposomes to release the reporters, and amplifying the reports by a suitable amplification technique (e.g., PCR). The low detection limit is achieved because DNA amplification is performed after as well as before binding to a biomarker. However, false results may be obtained due to various factors, such as contamination of samples, during the PCR.

Methods for amplifying signals using metal nanoparticles as a catalyst instead of enzymes have been proposed (U.S. Pat. No. 6,417,340 and T. A. Taton, C. A. Mirkin, R. L. Letsinger, Science 2000, 289, 1757-1760). According to these methods, gold nanoparticles act as a catalyst to reduce silver ions (Ag⁺) to silver (Ag), which is precipitated on the gold nanoparticles. The silver precipitate functions as another catalyst to allow continuous precipitation of silver around the gold nanoparticles, resulting in an increase in the size of the nanoparticles. The concentrations of biomarkers may be measured with high sensitivity through changes in color (T. A. Taton, C. A. Mirkin, R. L. Letsinger, Science 2000, 289, 1757-1760), electrical properties (S.-J. Park, T. A. Taton, C. A. Mirkin, Science 2002, 295, 1503-1506), and Raman spectrum (Y. C. Cao, R. Jin, C. A. Mirkin, Science 2002, 297, 1536-1540), which are attributed to the precipitation of the silver. Since the growth of the nanoparticles through the precipitation of the silver is limited to a maximum 30 nm, there is a limitation in lowering the detection limits for the biomarkers.

The above-mentioned methods developed hitherto are associated to a reduction in the detection limit of biosensors. Although some of the methods are preferred in terms of detection limit, they are not appropriate to apply to biosensors due to their low reliability and poor reproducibility. In addition, the problems of the methods are that complicated manufacturing procedure of biosensors and complex measurement procedures are involved. Thus, there is a need for a biosensor that has a low detection limit, high reliability and superior reproducibility. There is also a need for a biosensor that can be manufactured in a simple manner and allows simple measurement.

### SUMMARY OF THE INVENTION

It is one object of the present invention to provide a biosensor that has high reliability, superior reproducibility and a low detection limit, is manufactured in a simple manner, and allows simple measurement.

It is another object of the present invention to provide a biosensor that amplifies a substance having an amine group formed by the action of a nanocatalyst to induce large electrochemical, chromatic or fluorescent changes, or amplifies an electrochemical signal by redox cycling of the substance having an amine group formed by the action of a nanocatalyst to enable high-sensitivity measurement within a short time.

### BRIEF DESCRIPTION OF THE DRAWINGS

The above and other objects, features and other advantages of the present invention will be more clearly understood from the following detailed description taken in conjunction with the accompanying drawings, in which:

FIG. 1 is a schematic diagram of a nanocatalyst-based biosensor according to the present invention;

FIG. 2 shows schematic diagrams illustrating the use of labels in a biosensor using biospecific binding;

FIG. 3a shows a nanocatalyst in the form of a nanoparticle composed of one or more kinds of metal atoms, FIG. 3b shows a nanocatalyst including a nanoparticle having pinholes and metal nanoparticles, and FIG. 3c shows a nanocatalyst including a nanoparticle and metal cations present within the nanoparticle;

FIG. 4 shows substrates having a functional group used in the present invention, each of which is reduced to a corresponding substance having an amine group;

FIG. 5 shows the structures and formation reactions of benzene derivatives having an amine group;

FIG. 6 is a schematic diagram of an electrochemical nanocatalyst-based biosensor according to the present invention;

FIG. 7 shows reactions that can be induced in an electrochemical biosensor of the present invention;

FIG. 8 shows a graph of absorbance versus time upon reduction of p-nitrophenol, which is obtained in a Tris buffer solution (pH 9.0), using a gold nanocatalyst, and a graph of reaction rate as a function of the concentration of p-nitrophenol;

FIG. 9 shows a graph of absorbance versus time upon reduction of p-nitrophenol, which is obtained in a Tris buffer solution (pH 9.0), using an antibody-covered gold nanocatalyst, and a graph of reaction rate as a function of the concentration of the p-nitrophenol;

FIG. 10 is a schematic diagram of a sandwich-type electrochemical biosensor for measuring the concentration of prostate specific antigens (PSA) or mouse antibodies by using gold nanoparticles as labels;

FIG. 11 shows cyclic voltammograms for p-nitrophenol and p-aminophenol;

FIG. 12 shows cyclic voltammograms of a biosensor according to the present invention in the absence and presence of mouse antibodies;

FIG. 13 is a graph showing peak currents at different concentrations of mouse antibodies; and

FIG. 14 is a graph showing peak currents at different concentrations of PSA.

### DETAILED DESCRIPTION OF THE PREFERRED EMBODIMENTS

The above objects and other advantages of the present invention will be better understood by those skilled in the art from the following preferred embodiments with reference to the accompanying drawings.

According to the present invention, there is provided a biosensor for measuring the concentration of a biomarker in which a nanocatalyst label with a plurality of active sites catalyzes the reduction of a substrate to a substance having an amine group in the presence a reducing agent. In an embodiment of the present invention, the reduction is repeated to amplify the substance having an amine group, and electrochemical, chromatic or fluorescent changes after the reduction to the substance having an amine group are measured. That is, in the biosensor of the present invention, an organic substance as a substrate is reduced to a substance having an amine group by the catalysis of a nanocatalyst label with a plurality of active sites and the oxidation of a reducing agent, the reduction is continuously carried out to amplify the substance having an amine group, and the amount of the substance having an amine group is measured by taking advantage of the fact that the substance having an amine group exhibits different electrochemical, chromatic or fluorescent properties from those of the substrate, so that the concentration of a biomarker is measured with high sensitivity within a short time.

The nanocatalyst label used in the biosensor of the present invention may be i) in the form of a metal nanoparticle composed of one or more kinds of metal atoms, ii) a nanocatalyst including nanoparticles having large pinholes and one or more metal nanoparticles, each of which is composed of one or more kinds of metal atoms, present within the nanoparticles, or iii) a nanocatalyst including nanoparticles having large pinholes and one or more kinds of metal cations present within the nanoparticles.

Elements of the metal atoms and the metal cations may be selected from the group consisting of gold (Au), silver (Ag), platinum (Pt), palladium (Pd), rhodium (Rh), iridium (Ir), nickel (Ni), iron (Fe) and copper (Cu).

The reducing agent may be selected from the group consisting of NaBH₄, NaBH₃CN, hydrazine, formic acid, salts of formic acid, ammonia, hydrogen, and mixtures thereof.

The substrate may be a substance having at least one functional group selected from the group consisting of nitro, nitroso, azo, hydrazo, nitrile, azide, diazonium, hydroxylamine, and secondary amines.

The substance having an amine group may be an aminobenzene derivative or an aminomethylbenzene derivative.

In another embodiment of the present invention, there is provided an electrochemical biosensor that converts an electroinactive substrate into an electroactive substance having an amine group by means of a nanocatalyst, electrochemically oxidizes the substance having an amine group in an electrode, reduces the electrochemically oxidized substance by means of a reducing agent present in a solution, electrochemically reoxidizes the substance having an amine group, and repeats the above procedure to amplify an oxidation current.

The substrate that is electrochemically activated by means of the nanocatalyst may be p-nitrophenol, p-nitrosophenol, p-hydroxyphenylazide, p-diazoniumphenol, p-hydroxyphenylhydroxylamine, p-hydroxyphenyl p-sulfonylphenyl hydrazo, or p-hydroxyphenyl p-sulfonylphenyl azo.

The present invention will now be described in detail with reference to the accompanying drawings.

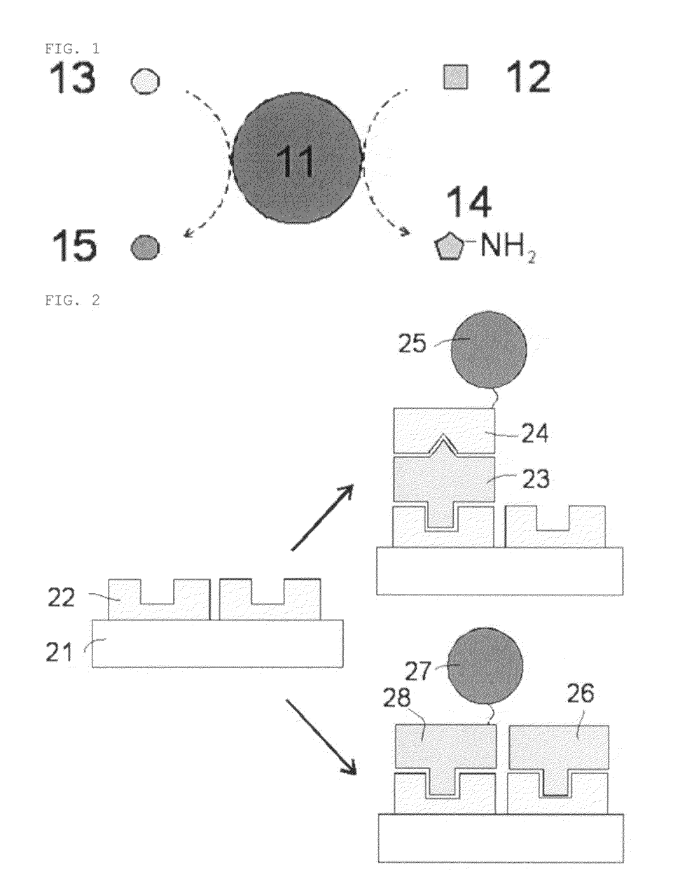

FIG. 1 is a schematic diagram of a biosensor according to the present invention. A reduction (or an oxidation) reaction using a catalyst always requires the use of a reducing agent (or an oxidant). As shown in FIG. 1, a substrate 12 is reduced by the action of a nanocatalyst 11 to form a substance 14 having an amine group, and a reducing agent 13 is oxidized to form an oxidized substance 15. Since the reduction reaction is very slow in the absence of the nanocatalyst 11, the overall reactions do not substantially occur. Meanwhile, the presence of the nanocatalyst 11 makes the overall reactions proceed continuously, leading to an increase in the concentration of the substance 14 having an amine group.

Some biosensors use enzymes as catalysts. One enzyme typically has only one active site. In contrast, one nanoparticle of the nanocatalyst 11 used in the present invention has a plurality of active sites. Accordingly, the reaction rate is increased due to the presence of the plurality of active sites in the nanoparticle although the reaction rate per active site of the nanoparticle is slow. As the size of the nanoparticle increases, the number of active sites per nanoparticle increases, making the reaction rate per nanoparticle faster.

FIG. 2 shows schematic diagrams illustrating how to use nanocatalyst labels in a biosensor for measuring the concentration of a biomarker using biospecific binding. Antibodies or biomolecules 22 are immobilized on the surface of a solid 21, such as an electrode, a nanoparticle or a bead, and the biomarker 23 is biospecifically bound thereto. Thereafter, another antibody or biomolecule 24 is biospecifically bound to the biomarker 23. A nanocatalyst 25 as a label is attached to the biomolecule 24. The antibody or biomolecule 24, to which the label is attached, is bound to the biomarker only when the biomarker 23 is present on the surface of the antibodies or biomolecules 22. The amount of the antibody or biomolecule 24, to which the label is attached, is dependent on the amount of the biomarker 23 present in a sample. Accordingly, the amount of the nanocatalyst 25 present on the surface of the antibody or biomolecule 24 affects the amount of a substance having an amine group, which is formed by the catalysis of the nanocatalyst. The amount of the biomarker 23 can be indirectly determined by the measuring the amount of the substance having an amine group. The biosensor of the present invention may be the sandwich-type biosensor shown in FIG. 2.

The biosensor of the present invention may be a biosensor using a particular reaction, such as a competitive reaction or displacement. A biomarker 26 and another biomarker 28, to which a nanocatalyst 27 is attached as a label, are biospecifically bound to the antibodies or biomolecules 22 through a competitive reaction or displacement. An increase in the amount of the nanocatalyst 27 present on the surface of the biomarker 28 indicates the presence of a smaller amount of the biomarker 26. Accordingly, the amount of a substance having an amine group formed by the catalysis of the nanocatalyst 27 decreases with increasing amount of the biomarker 26. Based on this principle, the amount of the biomarker 26 can be measured. The biomarkers 23 and 26 may be selected from DNA, RNA, proteins, organic substances, and the like.

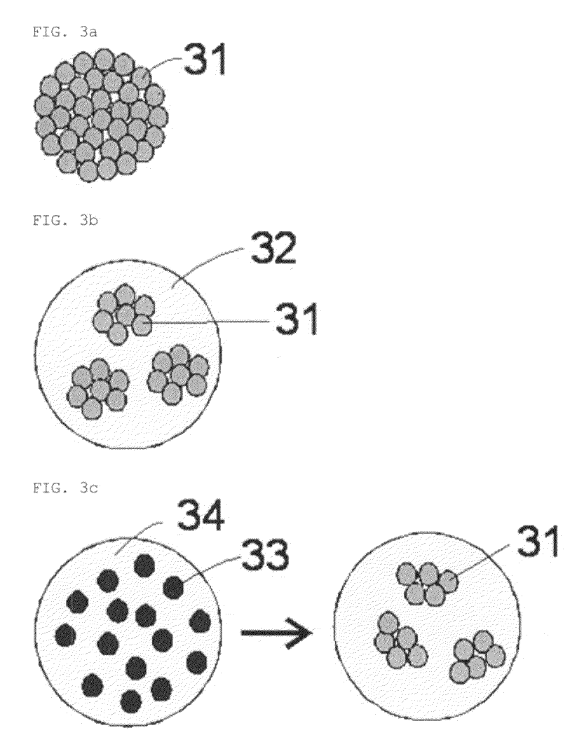

FIGS. 3a, 3b and 3c are schematic diagrams showing various structures of the nanocatalyst. As shown in FIG. 3a, the nanocatalyst may be in the form of a nanoparticle composed of one or more kinds of metal atoms 31. Examples of the metal atoms include gold, silver, platinum, palladium, rhodium, iridium, nickel, iron and copper atoms. As the size of the metal nanoparticle increases, the number of the metal atoms exposed to the surface of the metal nanoparticle is increased. Antibodies or biomolecules, which are biospecifically bound to a biomarker, are bound to the surface of the metal nanoparticle. Although the metal nanoparticle is surrounded by the biomolecules, it can function as a catalyst so long as a substrate and a substance having an amine group pass through small pinholes between the biomolecules.

As shown in FIG. 3b, the nanocatalyst may include a nanoparticle (e.g., a polymer or dendrimer nanoparticle) having large pinholes and a plurality of metal nanoparticles present within the nanoparticle. The plurality of metal nanoparticles present within the nanoparticle having large pinholes function as individual nanocatalysts. The metal nanoparticles can continuously function as nanocatalysts if a substrate and molecules having an amine group can readily migrate inside and outside the nanoparticle having large pinholes, such as a polymer or dendrimer nanoparticle.

FIG. 3c is a schematic diagram illustrating the use of a nanocatalyst including a nanoparticle 34 having large pinholes and metal cations 33 present within the nanoparticle 34. When the metal cations 33 present within the nanoparticle are strongly bound to the nanoparticle in a solution, a reducing agent for the catalysis of the nanocatalyst is added to reduce the metal cations to corresponding metal atoms 31, i.e., metal nanoparticles. The metal nanoparticles thus formed also function as individual nanocatalysts. Accordingly, the metal nanoparticles acting as nanocatalysts are formed by the addition of the reducing agent to operate a sensor, thus eliminating the need to previously form metal nanoparticles. As an element of the metal cations, there may be used, for example, gold, silver, platinum, palladium, rhodium, iridium, nickel, iron or copper.

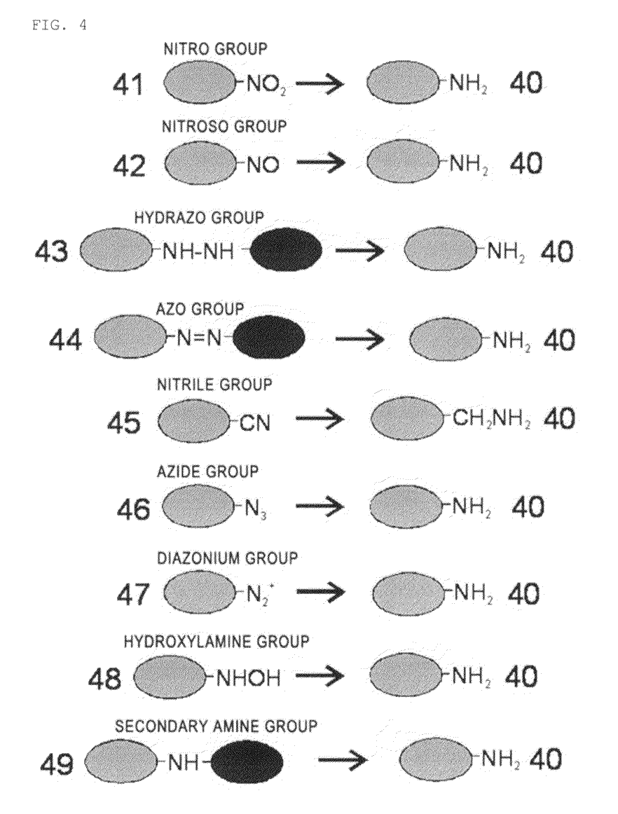

FIG. 4 shows reactions in which substrates are converted into corresponding substances having an amine group 40 by the reduction of the nanocatalyst. Specifically, a substrate 41 having a nitro group, a substrate 42 having a nitroso group, a substrate 43 having a hydrazo group, a substrate 44 having an azo group, a substrate 45 having a nitrile group, a substrate 46 having an azide group, a substrate 47 having a diazonium group, a substrate 48 having a hydroxylamine group, and a substrate 49 having a secondary amine group are reduced to corresponding substances 40 having an amine group. The reduction is carried out in a solution containing at least one reducing agent selected from NaBH₄, NaBH₃CN, hydrazine, formic acid, salts of formic acid, ammonia and hydrogen in the presence of the nanocatalyst.

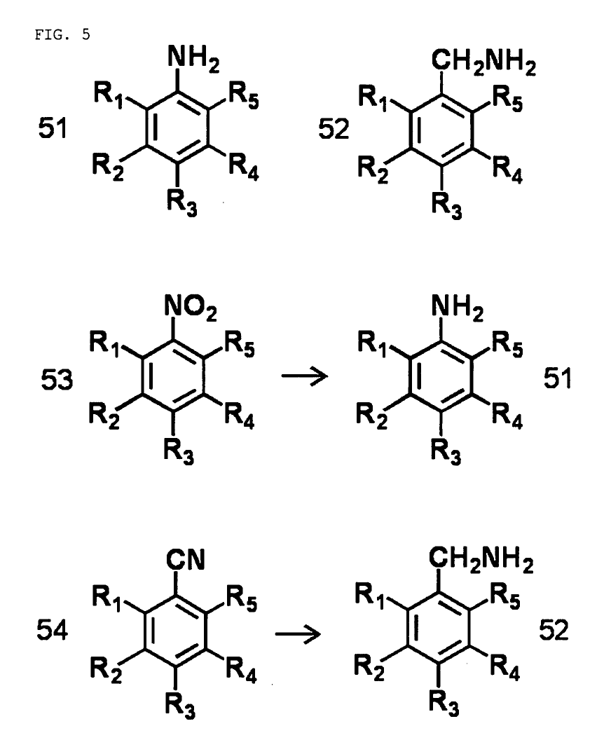

FIG. 5 shows the structures and formation reactions of benzene derivatives having an amine group. The amine group acts as a strong electron donating group. In the case where a substrate having an electron withdrawing group is reduced, significant changes in the ability of the functional group to donate or withdraw electrons are induced before and after the reduction. When the functional group is attached in close proximity to a benzene ring, significant changes occur in the energy levels of the highest occupied molecular orbital and the lowest unoccupied molecular orbital before and after the reduction. Such changes in energy level greatly affect the color, standard reduction potential and fluorescent properties of the benzene derivatives. For example, p-nitrophenol has a color in an aqueous solution, but p-aminophenol, a reduction product of p-nitrophenol, is colorless in an aqueous solution.

Examples of substances that have an amine group attached close to a benzene ring include aminobenzene derivatives 51 and aminomethylbenzene derivatives 52. In these derivatives, R₁, R₂, R₃, R₄ and R₅ may be each independently H, OH, NH₂ or an alkyl group, or may be bonded together to form a fused form of two or more benzene groups. In many cases, reduction of substances induces significant changes in the energy level of the substances. For example, a benzene derivative 53 having a nitro group as a strong electron withdrawing group is reduced to the aminobenzene derivative 51 having a strong electron donating group. As another example, a benzene derivative 54 having a cyano group as a strong electron withdrawing group is reduced to the aminomethylbenzene derivative 52 having an electron donating group. Various characteristics, such as color, standard reduction potential, fluorescent properties, of the substrates 53 and 54 before the reduction are greatly different from those of the benzene derivatives 51 and 52 having an amine group after the reduction. Therefore, these differences in characteristics before and after reduction can be used to increase the amplification of signals in sensors.

FIG. 6 is a schematic diagram of an electrochemical nanocatalyst-based biosensor according to the present invention. An electroinactive substrate 62 is electrochemically activated and is converted into a substance 64 having an amine group by a nanocatalyst 61, and a reducing agent 63 is converted into an oxidized substance 65. The electroinactive substrate 62 refers to a substance that can be electrochemically oxidized but whose oxidation does not readily occur in a typical potential range employed in the field of electrochemical biosensors due to its very high standard reduction potential. The substance 64 that is electroactive and has an amine group refers to a substance that is readily oxidized in a typical potential range. The electroinactive substrate 62 is continuously converted into the electroactive substance 64 by the nanocatalyst 61, and after the passage of a predetermined time, the electroactive substance 64 can be oxidized in an electrode 66 to generate a high current. The electroactive substance 64 is oxidized in the electrode 66 to provide electrons to the electrode 66, and is then converted into an oxidized substance 67. No reaction occurs after the electroactive substance 64 is converted into the oxidized substance 67 in general electrochemical biosensors, whereas the oxidized substance 67 is again converted into the electroactive substance 64 by the reducing agent 63 in the present invention. A large amount of the highly reactive reducing agent present in a solution serves to convert the oxidized substance 67 into the electroactive substance 64 as soon as the oxidized substance 67 is formed. This reaction also plays a role in oxidizing the reducing agent 63 to the oxidized substance 65. When the reducing agent 63 is present in a sufficiently large amount and a potential is continuously applied to the electrode 66 to oxidize the electroactive substance 64, mutual conversion between the electroactive substrate 64 and the oxidized substance 67 occurs continuously. This redox cycling increases the oxidation current measured in the electrode 66. The redox cycling can be achieved using one electrode 66 and one nanocatalyst 61. The electrochemical biosensor of the present invention has a high signal-to-noise ratio with increasing current and a high sensitivity.

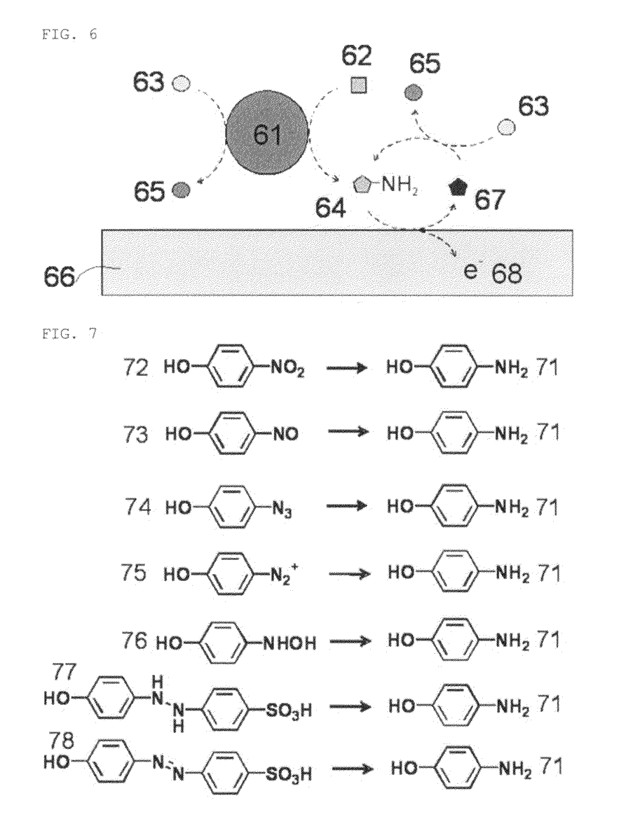

FIG. 7 shows reactions of substrates that may occur in the electrochemical biosensor of the present invention. Electrochemical oxidation of electroactive p-aminophenol, a reduction product of the substrates, readily occurs, whereas oxidation of the electroinactive substrates before reduction does not readily occur. That is, p-nitrophenol 72, p-nitrosophenol 73, p-hydroxyphenylazide 74, p-diazoniumphenol 75, p-hydroxyphenylhydroxylamine 76, p-hydroxyphenyl p-sulfonylphenyl hydrazo 77 and p-hydroxyphenyl p-sulfonyl phenyl azo 78 as the substrates are reduced to form electroactive p-aminophenol 71.

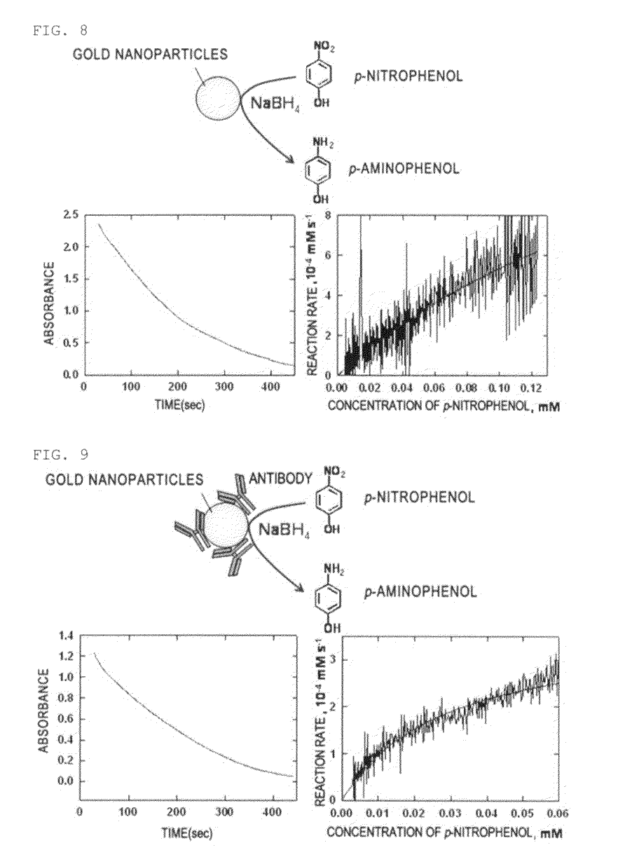

FIG. 8 shows a graph of absorbance versus time upon reduction of p-nitrophenol, which is obtained in a Tris buffer solution (pH 9.0), using gold nanoparticles as a nanocatalyst, and a graph of reaction rate as a function of the concentration of p-nitrophenol. The concentrations of the p-nitrophenol, the gold nanocatalyst and NaBH₄ are 0.2 mM, 126 pM and 10 mM, respectively. p-Nitrophenol strongly absorbs light at 400 nm, whereas p-aminophenol does not substantially absorb light at 400 nm. The reduction of the p-nitrophenol with time can be identified by measuring the absorbance at 400 nm over time. The gold nanoparticles, which have a size of 10 nm and are stabilized by citrate, are used as a nanocatalyst and an excess of NaBH₄ is used as a reducing agent to reduce the p-nitrophenol to p-aminophenol. The absorbance decreases with increasing time, and the measured absorbance values are expressed with respect to the concentrations of the p-nitrophenol to calculate the reaction rates at the respective concentrations.

The reaction rate of a general enzyme can be described by the Michaelis-Menten mechanism, which requires the use of the initial rate method by which the reaction rate is measured in a state where the concentration of a product is zero to exclude the influence of the product on a reactant. In the reduction of p-nitrophenol, the reaction rate of the reactant is not affected by the product (i.e. p-aminophenol). Since the reverse reaction of the reduction does not substantially occur and the forward reaction only occurs, the reaction rate measured at a concentration of the p-nitrophenol during formation of the p-aminophenol can be considered as an initial reaction rate at the concentration. As a result, the turnover number (k_{cat}) and the catalytic efficiency (k_{cat}/K_{M}) of the gold nanocatalyst can be calculated from the graph of FIG. 8 showing variations in reaction rate with varying concentrations of the p-nitrophenol. The gold nanocatalyst is calculated to have a very high turnover number of 1.6±0.5×10⁴ s⁻¹ and a very high catalytic efficiency of 6.9±0.8×10⁷ M⁻¹s⁻¹. These results are due to the fact that the catalysis of the gold nanocatalyst is very fast and a plurality of active sites are present in each of the gold nanocatalyst particles.

FIG. 9 shows a graph of absorbance versus time upon reduction of p-nitrophenol, which is obtained in a Tris buffer solution (pH 9.0), using an antibody-covered gold nanocatalyst as a nanocatalyst, and a graph of reaction rate as a function of the concentration of the p-nitrophenol. The concentrations of the p-nitrophenol, the gold nanocatalyst and NaBH₄ are 0.1 mM, 505 pM and 10 mM, respectively. The antibody-covered gold nanocatalyst is calculated to have a turnover number of 1.5±0.7×10³ s⁻¹ and a catalytic efficiency of 1.9±0.4×10⁷ M^{31 1}s⁻¹, which are slightly lower than those of the uncovered gold nanocatalyst. However, the antibody-covered gold nanocatalyst exhibits very excellent catalytic characteristics. Although antibodies are adsorbed on the surface of the gold nanoparticles, the p-nitrophenol and p-aminophenol readily migrate through pinholes between the antibodies to allow the antibody-covered gold nanoparticle to have a high turnover number and a high catalytic efficiency.

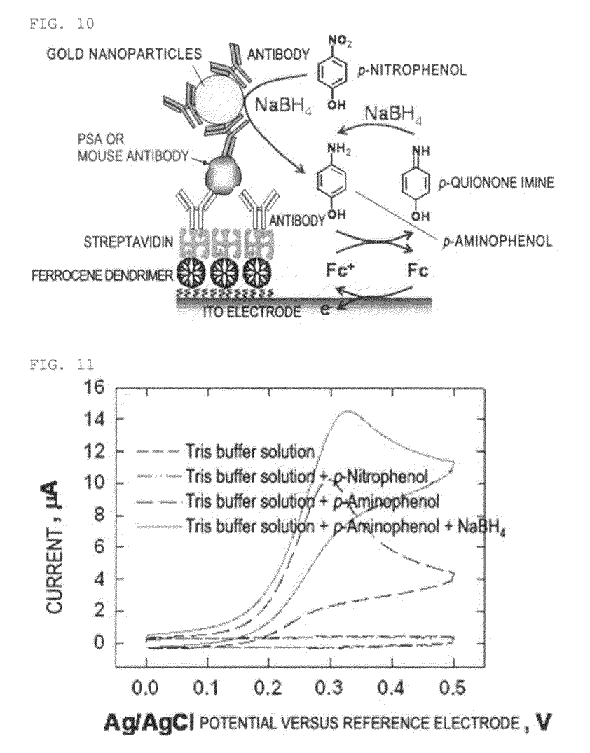

FIG. 10 is a schematic diagram of a sandwich-type electrochemical biosensor for measuring the concentration of prostate specific antigens (PSA) or mouse antibodies by using gold nanoparticles as labels. An indium thin oxide (ITO) electrode is covered with a ferrocene dendrimer to facilitate the transfer of electrons to the surface of the electrode. For the immobilization of biotin-linked antibodies on the ferrocene dendrimer, streptavidin is formed on the ferrocene dendrimer. In a state in which anti-PSA antibodies (anti-PSA IgG) or anti-mouse antibodies (anti-mouse IgG) are immobilized on the surface of the ferrocene dendrimer through biotin-streptavidin linkages, the antibodies are bound to PSA or mouse antibodies present in a sample. The PSA or mouse antibodies are bound to gold nanoparticles covered with new anti-PSA IgG or anti-mouse IgG. The gold nanoparticles cause catalysis. The ferrocene dendrimer has a structure in which 0.5% of 64 amine groups of a dendrimer are linked to a ferrocene.

p-Nitrophenol is converted into p-aminophenol by the action of the gold nanocatalyst and NaBH₄ as a reducing agent. The p-aminophenol is allowed to be formed in the largest amount and is then electrochemically oxidized with the help of the ferrocene to form p-quinone imine. NaBH₄ acts to convert the p-quinone imine into p-aminophenol, which may be electrochemically reoxidized. This redox cycling increases the oxidation current measured in the electrode.

FIG. 11 shows cyclic voltammograms for p-nitrophenol and p-aminophenol. The cyclic voltammograms are recorded using an ITO electrode covered with a ferrocene dendrimer in a Tris buffer solution (pH 9.0). The cyclic voltammograms demonstrate that the oxidation current of a solution containing p-nitrophenol solution is similar to that of the solution containing no p-nitrophenol. In contrast, a high current is observed by the oxidation of p-aminophenol present in the solution. That is, p-nitrophenol is electroinactive, while p-aminophenol, a reduction product of p-nitrophenol, is electroactive. Accordingly, the signal of p-aminophenol can be measured even without a background current of p-nitrophenol.

The peak current measured in the presence of NaBH₄ is higher than the peak current measured in the absence of NaBH₄. This is because p-quinone imine is reduced to p-aminophenol by the reducing agent NaBH₄ and the p-aminophenol is reoxidized on the electrode i.e., redox cycling occurs.

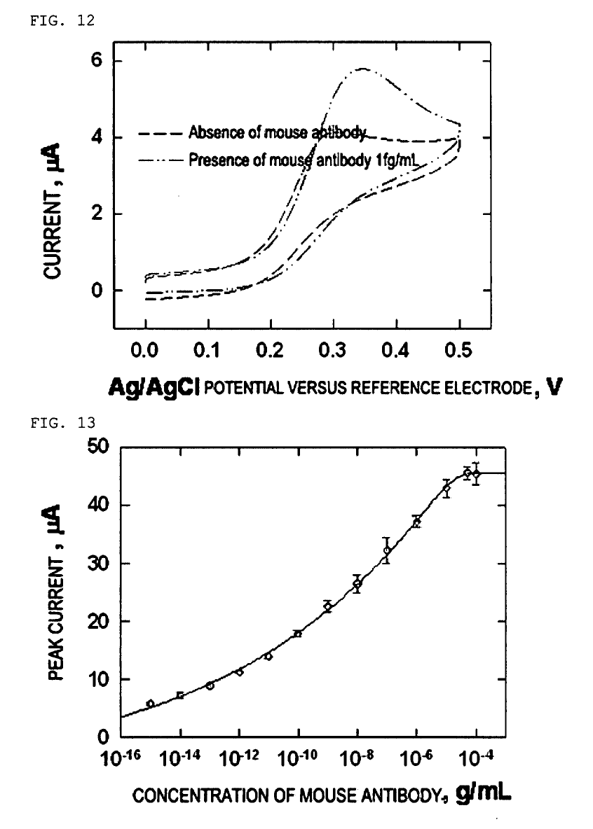

FIG. 12 shows cyclic voltammograms of the biosensor in the absence and presence of mouse antibodies. The peak current of the biosensor is 4.5±0.5 μA in the absence of mouse antibodies, and that of the biosensor is 6.0±0.4 μA in the presence of mouse antibodies at a concentration of 1 fg/mL. As is evident from these results, the detection limit of the biosensor for the mouse antibodies is a minimum of 1 fg/mL.

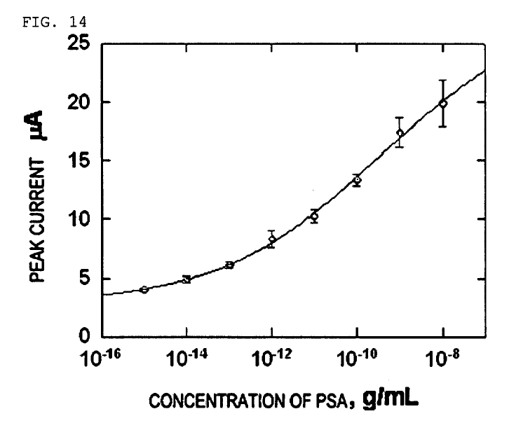

FIG. 13 is a graph showing peak currents at different concentrations of the mouse antibodies. The detection limit of the biosensor for the mouse antibodies is as low as 1 fg/mL. Taking into consideration the molecular weight (150,000) of the antibodies, a concentration of 1 fg/mL corresponds to a detection limit of 7 aM. The reason why the low detection limit for the mouse antibodies is achieved is because p-nitrophenol is reduced at a high rate by the nanocatalyst and the current signal is amplified by the feedback. As can be seen from the graph shown in FIG. 13, the concentration of the mouse antibodies can be measured in a very broad range of 1 fg/mL to 10 μg/mL. Another advantage of the biosensor according to the present invention is a very broad detection range.

FIG. 14 is a graph showing peak currents at different concentrations of the PSA. The detection limit of the biosensor for the PSA is as low as 1 fg/mL.

Those skilled in the art will appreciate that various modifications, additions and substitutions are possible, without departing from the technical spirit of the invention as disclosed in the accompanying claims. Therefore, the foregoing embodiments and the accompanying drawings do not serve to limit the scope of the present invention.

As apparent from the above description, the biosensor of the present invention uses a nanocatalyst as a label instead of an enzyme that is mainly used in a conventional biosensor. The biosensor of the present invention has a low detection limit. Optionally, the biosensor of the present invention can be used to measure the concentration of a biomarker in a very broad range.

Since the biosensor of the present invention can induce significant electrochemical, chromatic or fluorescent changes by the catalysis of the nanoparticle, it allows simple measurement and has high sensitivity.

In addition, the biosensor of the present invention can be utilized as a key technology in immunoassay for the analysis of antigens or antibodies and DNA sensors for the analysis of DNA.

What is claimed is:

1. A biosensor for measuring the concentration of a biomarker comprising:
a nanocatalyst label with a plurality of active sites catalyzes the reduction of a substrate to a substance having an amine group in the presence of a reducing agent.

2. The biosensor according to claim 1, wherein the reduction is repeated to amplify the substance having an amine group, and electrochemical, chromatic or fluorescent changes after the reduction to the substance having an amine group are measured.

3. The biosensor according to claim 1, wherein the substrate is electroinactive, the substance having an amine group is electroactive, and the oxidation current is amplified by the redox cycling of the substance having an amine group by the reducing agent.

4. The biosensor according to claim 1, wherein the nanocatalyst label is i) in the form of a metal nanoparticle composed of metal atoms, ii) a nanocatalyst including nanoparticles having pinholes and metal nanoparticles present within the nanoparticles, or iii) a nanocatalyst including nanoparticles having pinholes and metal cations present within the nanoparticles.

5. The biosensor according to claim 4, wherein the elements of the metal atoms and the metal cations are selected from the group consisting of gold, silver, platinum, palladium, rhodium, iridium, nickel, iron, and copper.

6. The biosensor according to claim 1, wherein the reducing agent is selected from the group consisting of NaBH₄, NaBH₃CN, hydrazine, formic acid, salts of formic acid, ammonia, hydrogen, and mixtures thereof.

7. The biosensor according to claim 1, wherein the substrate is a substance having at least one functional group selected from the group consisting of nitro, nitroso, azo, hydrazo, nitrile, azide, diazonium, hydroxylamine, and secondary amines.

8. The biosensor according to claim 2, wherein the substance having an amine group is an aminobenzene derivative or an aminomethylbenzene derivative.

9. The biosensor according to claim 3, wherein the substrate is selected from the group consisting of p-nitrophenol, p-nitrosophenol, p-hydroxyphenylazide, p-diazoniumphenol, p-hydroxyphenylhydroxylamine, p-hydroxyphenyl p-sulfonylphenyl hydrazo, p-hydroxyphenyl p-sulfonylphenyl azo, and mixtures thereof.
